(19) United States
(12) Patent Application Publication
(10) Pub. No.: US 2025/0205486 A1
(43) Pub. Date: Jun. 26, 2025

(54) SYSTEMS AND METHODS FOR A WEARABLE, REAL-TIME COGNITIVE BEHAVIORAL THERAPY DEVICE

(71) Applicant: THE BOARD OF TRUSTEES OF THE LELAND STANFORD JUNIOR UNIVERSITY, Stanford, CA (US)

(72) Inventors: Casey Harrison Halpern, Philadelphia, PA (US); Camarin Rolle, Novato, CA (US)

(21) Appl. No.: 18/395,256

(22) Filed: Dec. 22, 2023

Related U.S. Application Data
(63) Continuation of application No. PCT/US22/35842, filed on Jun. 30, 2022.
(60) Provisional application No. 63/217,077, filed on Jun. 30, 2021.

(51) Int. Cl. A61N 1/36

(52) U.S. Cl. CPC A61N 1/36025; A61N 1/36031

(57) ABSTRACT

Devices, systems, and methods for a wearable, real-time cognitive behavioral detection and/or therapy device for detecting impulsivity states of a user, and for providing alerts to the patient and/or a patient-identified network of persons, of an impending impulsivity state. The device utilizes a combination of wearable, non-invasive, sensors configured to be worn by a user and to detect electrophysiology signals and/or psychophysiological signals of the user. The sensors output respective sensor signals corresponding to the electrophysiology signals and psychophysiological signals and transmit the respective sensor signals to a computing device. The computing device has a software application which programs the computing device to process the sensor signals and provide informational and/or therapeutic information regarding an impulsivity state of the user. The device may also include a system for delivering electrical stimulation directly to the user in response to the impulsivity state detected by the device.

### RELATED APPLICATION DATA

The present application is a continuation of International Application No. PCT/US2022/035842, filed Jun. 30, 2022, which claims benefit of U.S. provisional application Ser. No. 63/217,077, filed Jun. 30, 2021, the entire disclosures of which are expressly incorporated by reference herein.

### TECHNICAL FIELD

The present invention relates to wearable medical devices, and, more particularly, to systems and methods for a wearable, real-time cognitive behavioral therapy device.

### BACKGROUND

Impulsivity is one of the most pervasive and disabling behavioral disorders common to many disorders of the brain. Heightened responsivity in the nucleus accumbens (NAc) during anticipation of a rewarding stimulus predisposes to impulsive behavior, which can have severe implications for development of maladaptive behaviors. Notably, electrophysiological, neurochemical, and functional neuroimaging correlates have been reported in multiple species during brief windows of anticipation. These correlates (or biomarkers) that precede a “moment of weakness” have potential to inform a therapeutic to deliver a time-sensitive intervention.

Reward hypersensitization is a common feature of neuropsychiatric disorders, manifesting as impulsivity for anticipated incentives. Temporally specific changes in activity within the nucleus accumbens (NAc), which occur during anticipatory periods preceding consummatory behavior, represent a critical opportunity for intervention. However, no available therapy is capable of automatically sensing and therapeutically responding to this vulnerable moment in time when anticipation-related neural signals may be present.

### SUMMARY

Disclosed herein are innovative devices and methods for a wearable, real-time cognitive behavioral therapy device for detecting impulsivity states of a user, and for providing alerts to the patient and/or a patient-identified network of persons, of an impending impulsivity event once an activity threshold is surpassed. The methods and devices utilize a combination of wearable, non-invasive, sensors configured to be worn by a user and to detect electrophysiology signals at the scalp of the user and/or physiological signals of the user, such as at body location other than the scalp. For instance, the physiological signals may be psychophysiological signals (i.e., physiological signals related to mental processes or disorders). The sensors output respective sensor signals corresponding to the electrophysiology signals at the scalp and physiological signals at other than the scalp and transmit the respective sensor signals to a computing device. The computing device is configured to receive the sensor signals. The computing device has a software application (“app”) which programs the computing device to process the sensor signals and provide informational and/or therapeutic information regarding an impulsivity state of the user.

In additional aspects, the device may be configured to provide therapeutic treatment to the user, such as delivering electrical stimulation directly to the user in response to the impulsivity state detected by the device.

According to one disclosed embodiment, a wearable, cognitive behavioral therapy device includes a first sensor configured to be worn on a user and a second sensor configured to be worn on the user. The first sensor is a non-invasive sensor configured to detect an electrophysiology signal at the scalp of the user which is a correlate of a user's NAc signal. For example, the first sensor may be a scalp sensor comprising an array of scalp sensors which detects a dorsal-lateral prefrontal cortex (dlPFC) theta (4-8 Hz) signal. The first sensor is configured to output a first sensor signal corresponding to the detected electrophysiology signal. The second sensor is a non-invasive sensor configured to detect a physiological signal (e.g., heart rate, heart rate variability, etc.) which is a correlate of a user's NAc signal. For example, the second sensor may be a heart rate sensor comprising one or more electrodes which can be placed above the user's wrist to detect heart rate and heart rate variability. The second sensor is configured to output a second sensor signal corresponding to the detected physiological signal.

The device further includes a computing device in operable communication with the first sensor and second sensor. For example, the computing device may be a portable computing device, such as a smartphone, tablet computer, handheld computer, other portable computer, or the like. The computing device is configured to receive the first sensor signal and second sensor signal. The computing device also has an impulsivity software application (app) which programs the computing device to process the sensor signals and utilize a detection algorithm to detect an impulsivity state of the user. The app may then provide informational and/or therapeutic information regarding an impulsivity state of the user. For example, the app may push alert notifications to the user (also referred to as a “patient”), a patient-identified network of persons (e.g., clinicians, etc., providing treatment to the patient), to notify them of an impulsivity state of the patient.

In another aspect, the device may be specifically configured to treat patients with Loss of Control (LOC) eating disorders. It has been identified through invasive, empirical experimentation that the low-frequency (1-4 Hz) delta band signal of the NAc is a sensitive measure to impulsivity behaviors causing eating disorders. One such experiment is described in the publication, “Closing the loop on impulsivity via nucleus accumbens delta-band activity in mice and man”, Proceedings of the National Academy of Sciences of The United States of American (PNAS) Jan. 2, 2018, Jan. 2, 2018, vol. 115, no. 1 (hereinafter referred to as “the Closing the loop publication”); also see PCT publication WO2018/064225 to Halpern, the entire disclosure of which is incorporated by reference herein. All publication, patents and references cited herein are incorporated by reference herein in their entirety. Accordingly, in this aspect, the first sensor is a non-invasive sensor configured to detect a correlate of the delta band of NAc signal, for example, the dlPFC Theta signal. In the experiment described in the Closing the loop publication, the potential for the closed-loop system to intervene during a vulnerable period immediately preceding receipt of highly rewarding stimuli was examined. The finding that electrically stimulating the NAc in mice anticipating a food reward effectively attenuates binge-eating behavior is leveraged for use in treating humans. To “close the loop” on this intervention using an automatic stimulatory system, however, the identification, characterization, and refinement of an anticipatory biomarker are critical steps. Local field potential (LFP) recordings from the mouse and human NAc during a period of reward anticipation were made, and prominent delta oscillations elicited during anticipation of a highly rewarding stimulus were found. Multiunit analysis reveals strong correlations between delta oscillations and unit activities in the NAc. Utilizing a pre-determined power threshold, this translational biomarker was used as a trigger. As such, the closed-loop system blocked binge eating in mice with remarkable behavioral specificity, a result which may be utilized in targeted intervention for neuropsychiatric patients suffering from hypersensitivity to pathological motivations.

In another aspect of the device, the second sensor may be one or more electrodes for detecting the user's heart rate and heart rate variability. One or more correlates of the user's heart rate and heart rate variability to the NAc signal at the physiologic level (i.e., heart rate peaking, heart rate variations). The causality of this correlate is identified by NAc stimulation, such as measuring the correlate with NAc stimulation on and off. The sensitivity of the correlate may also be identified by state-manipulation.

The use of both the electrophysiological signal at the scalp and the physiological signal in determining the impulsivity state improves the specificity, sensitivity, and reliability of the device. For instance, the detection algorithm utilizing both the electrophysiological signal at the scalp and the physiological signal can be validated for specificity, sensitivity, and reliability in detecting an impulsivity state.

In another aspect, the computing device may be configured to communicate with the first sensor and second sensor via a wireless communication, such as Bluetooth, WiFi, or other suitable wireless communication system. Accordingly, the computing device, first sensor and second sensor each have a respective wireless communication module configured to communicate with the other communication modules, such as Bluetooth wireless communication modules, WiFi adapters, etc.

In another aspect, the device may further include an electrical stimulation system configured to deliver therapeutic electrical stimulation directly to the user in response to the impulsivity state detected by the device. In one aspect, the electrical stimulation system may include an electrical stimulator configured to be implanted into the user's brain to stimulate the NAc. The app utilizes a control program to control the electrical stimulation system to deliver a controlled, closed-loop electrical stimulation to the user based on the detected impulsivity state. The electrical stimulation may be configured to attenuate the impulsivity state. For example, in the case of an impending impulsivity event comprising a loss of control eating behavior, the electrical stimulation may be configured to attenuate the loss of control eating behavior. In another aspect, the electrical stimulation may be a brief train of high-frequency electrical stimulation pulses to the NAc.

In still another aspect, the device may be configured to detect and/or treat other impulsivity and anxiety based disorders for implementation of the sensor detection in disorders such as obsessive compulsive disorder (OCD), addiction, alcoholism, eating disorders, generalized anxiety order (GAD), post-traumatic stress disorder (PTSD), etc. The first sensor and second sensor are configured to detect respective correlates of brain signals related to impulsivity and anxiety based disorders. In still another aspect, the electrical stimulation system and control program may be configured to deliver electrical stimulation to the brain (e.g., the NAc) to attenuate the particular disorder, such as OCD, GAD, PTSD, etc.

Another embodiment disclosed herein is directed to methods of using the wearable, cognitive behavioral therapy device. According to one embodiment, the method includes obtaining a first sensor signal corresponding to a detected electrophysiology signal from a first sensor worn on the scalp of the user. A second sensor signal corresponding to a detected physiological signal is obtained from a second sensor positioned at a body location of the user other than the scalp. The computing device receives the first sensor signal and second sensor signal. Then, the computing device processes the first sensor signal and the second sensor signal and detects an impulsivity state of the user utilizing a detection algorithm which utilizes both the first sensor signal and the second sensor signal.

In additional aspects of the method, the method may include any one or more of the aspects, functions and features of the additional aspects of the wearable, cognitive behavioral therapy device described herein. For example, the method may further include delivering therapeutic electrical stimulation using the electrical stimulation system. In such case, the method also comprises the software application utilizing the control program to control the electrical stimulation system to deliver a controlled, closed-loop electrical stimulation to the user based on the impulsivity state. Then, the electrical stimulation system delivers the controlled, closed-loop electrical stimulation directly to the user in response to the impulsivity state.

Other aspects and features of the present invention will become apparent from consideration of the following description taken in conjunction with the accompanying drawings.

### BRIEF DESCRIPTION OF THE DRAWINGS

The foregoing and other aspects of exemplary embodiments are described in further detail with reference to the accompanying drawings, wherein like reference numerals refer to like elements (e.g., elements having the same number are considered like elements such as 50a and 50b) and the description for like elements shall be applicable for all described embodiments wherever relevant:

FIG. 1 is a schematic diagram of an exemplary embodiment of a wearable, real-time, cognitive behavioral therapy system.

FIG. 2 illustrates the placement of the scalp electrophysiological sensor of the system of FIG. 1 on a user.

FIG. 3 illustrates the placement of the physiological sensor of the system of FIG. 1 on a user.

FIG. 4 is a schematic illustration of an experimental methodology for determining a correlate of the delta signal of the NAc.

FIG. 5 illustrates results of the experiment of FIG. 4 in the form of a plot of all 64 scEEG channels showing that the theta signal (4-8 Hz), as detected by the non-invasive scalp EEG, is a correlate with the invasive delta signal of the left ventral NAc.

FIGS. 6-7 illustrate results of the experiment of FIG. 4 showing that the correlation between the non-invasively detected scalp EEG theta signal (4-8 Hz) and the invasive delta signal of the left ventral NAc is significantly elevated during “appetitive” anticipatory state (preceding milkshake), compared to “non-appetitive” anticipatory states (preceding water).

FIG. 8 illustrates results of the experiment of FIG. 4 for a test subject whose preference switched to no longer favor water over milkshake, which shows that the anticipatory elevation is not seen in the dlPFC theta signal specific to “appetitive” state.

FIG. 9 illustrates results of the experiment of FIG. 4 showing a T-Score comparison of the “appetitive” anticipatory state preceding milkshake compared to water.

FIG. 10 is a flow chart on an exemplary method of using the wearable, real-time, cognitive behavioral therapy system to detect and/or treat impulsivity states of a user.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

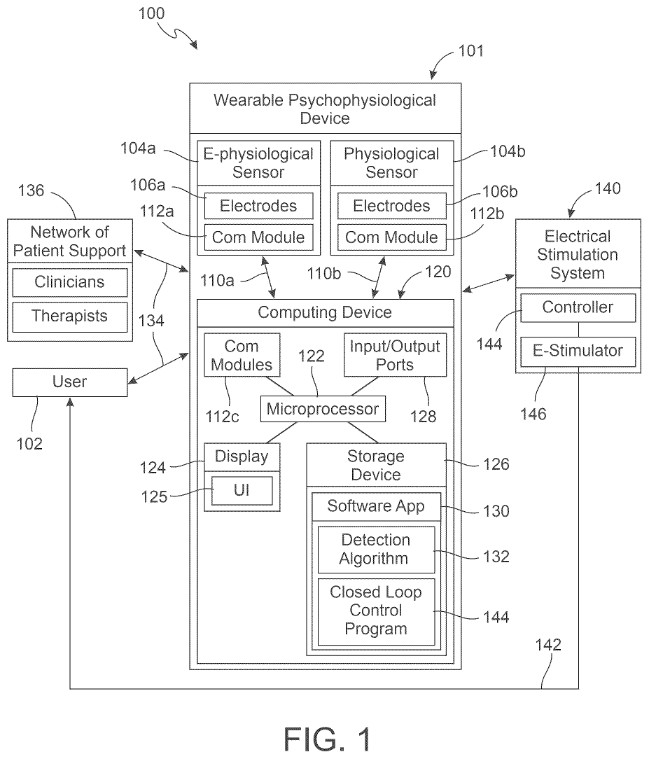

Turning to the drawings, FIG. 1 shows an exemplary embodiment of a wearable, real-time, cognitive behavioral therapy system 100 for detecting and/or treating impulsivity states of a user 102. The cognitive behavioral therapy system 100 includes a wearable, real-time, psychophysiological detection device 101 (also referred to as a “cognitive behavioral therapy device”), and an optional electrical stimulation system 140 operably coupled to the cognitive behavioral therapy system 100.

The psychophysiological detection device 101 includes a plurality of electrophysiological and physiological sensors 104 configured to be worn on the user 102. In the exemplary embodiment of FIG. 1, the device 101 includes two sensors 104, including a non-invasive scalp electrophysiological sensor 104a (i.e., a first sensor) configured to be worn on the user 102 and a non-invasive physiological sensor 104b (i.e., a second sensor) configured to be worn on the user 102. The device 101 may have additional sensors 104, such as one or more additional electrophysiological sensors 104a and/or physiological sensors 104b, for detecting various electrophysiological signals and/or physiological signals which are determined to be correlates of a brain signal related to impulsivity in humans.

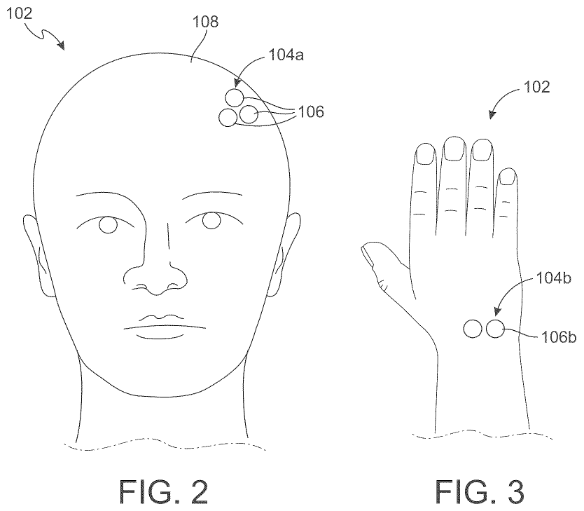

The scalp electrophysiological sensor 104a comprises a plurality of electrodes 106 (e.g., dry) which are configured to be placed above the cortex of the user on the user's scalp 108, as shown in FIG. 2. The scalp electrophysiological sensor 104a is configured to detect an electrophysiology signal 110a at the scalp of the user 102 which is a correlate of a user's NAc signal. In one embodiment, the scalp electrophysiological sensor 104a is configured to detect a dorsal-lateral prefrontal cortex (dlPFC) theta (4-8 Hz) signal which is a correlate of a delta signal of NAc (as described below), or any suitable brain signal for the particular behavioral disorder to be monitored and/or treated using the device. As described in the Closing the loop publication, empirical invasive experimentation has identified that the low-frequency (1-4 Hz) delta band signal of the NAc is a sensitive measure to impulsivity behaviors causing eating disorders. Additional experiments, described in more detail below, have also validated the specificity of a non-invasive target to identify scalp signals that show connectivity patterns over time with the delta signal of the NAc. The results of such experiments, described in more detail below, show that the theta signal (4-8 Hz) above the dlPFC, as detected by a scalp EEG, is a correlate with the invasive NAc delta signal during a task previously found to map onto LOC eating behaviors. Accordingly, in one embodiment, the scalp electrophysiological sensor 104a is a non-invasive sensor configured to detect the dlPFC Theta signal (i.e., the electrophysiological signal) correlate of the delta band of NAc signal.

The scalp electrophysiological sensor 104a outputs a scalp electrophysiological sensor signal 110a corresponding to the detected electrophysiological signal. The scalp electrophysiological sensor 104a includes a wireless communication module 112a configured to wirelessly transmit the scalp electrophysiological sensor signal 110a to a computing device 120 via a wireless communication module 112c of the computing device 120. The respective wireless communication modules 112 of the scalp electrophysiological sensor 104a, physiological sensor 104b and computing device 120 may be communication modules for any suitable wireless communication protocol, including Bluetooth, WiFi, wireless USB, etc. Alternatively, the scalp electrophysiological sensor 104a may be electronically coupled to the computing device 120, such as by a suitable bus or conductor connected to an input port 120 of the computing device 120.

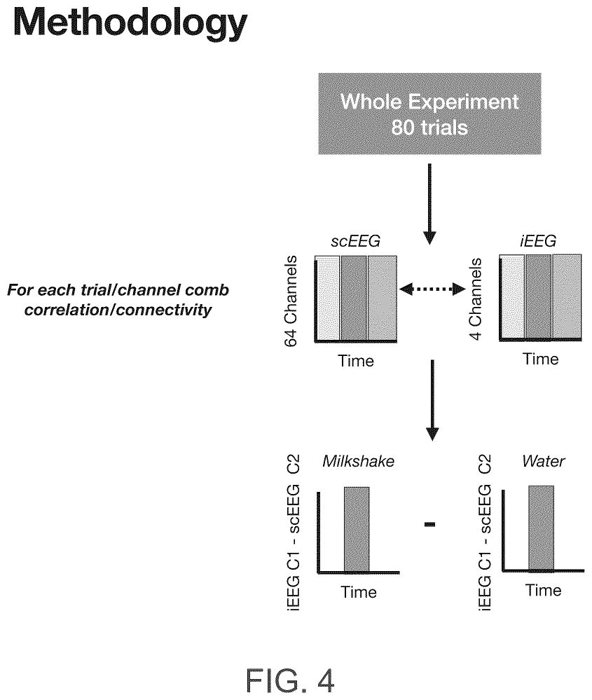

The physiological sensor 104b is a non-invasive sensor configured to detect one or more physiological signals. In this exemplary embodiment, the physiological signals are heart rate and heart rate variability. Heart rate and heart rate variability are determined to be correlates of a user's NAc signal. The physiological sensor 104b is a heart rate sensor comprising one or more electrodes 106b for placement above the user's wrist, as shown in FIG. 3, to detect heart rate and heart rate variability. The physiological sensor 104b outputs a physiological sensor signal 110b corresponding to the detected physiological signal. The causality of this correlate is identified by NAc stimulation, such as measuring the correlate with NAc stimulation on and off. The sensitivity of the correlate may also be identified by state-manipulation. The physiological sensor 104b includes a wireless communication module 112b configured to wirelessly transmit the physiological sensor signal 110b to the computing device 120 via the wireless communication module 112c of the computing device 120. Alternatively, the physiological sensor 104b may be electronically coupled to the computing device 120, such as by a suitable bus or conductor connected to an input port 120 of the computing device 120.

The exemplary computing device 120 is a portable computer and is in operable communication with the scalp electrophysiological sensor 104a and physiological sensor 104b. As some non-limiting examples, the computing device 120 is a smartphone, tablet computer, handheld computer, other portable computer, or the like. The computing device 120 has microprocessor 122, a display 124 (e.g., LCD, LED, etc.) for displaying a user interface 125, a storage device 126 (e.g., hard drive, SSD), and input/output ports 128. The computing device 120 also has a wireless communication module 112c configured to communicate with the wireless communication modules 112a, 112b of the scalp electrophysiological sensor 104a and physiological sensor 104b to receive the respective scalp electrophysiological sensor signal 110a and physiologic sensor signal 110b.

The computing device 120 also has an impulsivity software application (app) 130 which programs the computing device 120 to process the scalp electrophysiological sensor signal 110a and physiologic sensor signal 110b to detect an impulsivity state and/or deliver treatment for a behavioral disorder. The app 130 may be stored on the storage device 126 and includes a detection algorithm 132 to detect an impulsivity state of the user 102 based on the scalp electrophysiological sensor signal 110a and physiologic sensor signal 110b. For example, the detection algorithm may include one or more detection thresholds that can be used to detect an impulsivity state of the user using this electrophysiological scalp sensor that detects low frequency power specific to impulsiveness. For example, if the algorithm detects that information related to the sensed signals is above a threshold, the algorithm may make a determination that the user is in or about to be in a particular impulsivity state for which an alert and/or therapy is initiated. The app 130 also programs the computing device 120 to transmit informational and/or therapeutic information regarding the detected impulsivity state of the user 102. The app 130 may transmit alert notifications 134 to the user 102, a patient-identified network of persons 136 (e.g., clinicians, etc., providing treatment to the patient), to notify them of an impulsivity state of the patient 102. The alert notification 134 may be sent by any suitable means, such as via text message, audio message, audio alert, email, push notification, etc.

In certain embodiments, the system 100 (or device 101) optionally also includes an electrical stimulation system 140 configured to deliver electrical stimulation 142 directly to the user 102 in response to the impulsivity state detected by the wearable psychophysiological detection device 101. The electrical stimulation system 140 may be any suitable electrical stimulation system. The electrical stimulation system 140 is operably coupled to the computing device 120 via a suitable communication system, such as a suitable bus or wireless communication module. The electrical stimulation system 140 includes a controller 144 configured to control the operation of the electrical stimulation system 140, and an electrical stimulator 146 operably coupled to the controller 144. The electrical stimulator 146 comprises an electrical power source and one or more electrodes or other electrical stimulation elements for delivering electrical stimulation to an anatomy of the user 102 (e.g., brain anatomy). The app 130 of the computing device 130 utilizes a closed-loop control program 144 to control the electrical stimulation system 140 to deliver a controlled, closed-loop electrical stimulation 142 to the user 102 based on the detected impulsivity state. The electrical stimulation device 140 may be same or similar to the electrical stimulation device used in the RNS® System, available from NeuroPace, Inc.

The publication, “Brain-Responsive Neurostimulation for Loss of Control Eating: Early Feasibility Study”, Neurosurgery, Vol. 87, No. 6, December 2020, pp. 1277-1288 describes examples of using a closed-loop stimulation system to treat LOC eating disorders. The feasibility study described in this publication involves patients undergoing bilateral closed-loop stimulation of the NAc for LOC eating disorder. In one embodiment, the wearable, real-time, cognitive behavioral therapy system 100 may be configured to deliver electrical stimulation 142 via the electrical stimulation system 140 as described in this publication, for example, to treat LOC eating disorders or other impulsivity and anxiety based disorders.

In other embodiments, the wearable, real-time, cognitive behavioral detection system 100 and/or device 101 may be configured to detect and/or treat other impulsivity and anxiety based disorders for implementation of the sensor detection in disorders such as obsessive compulsive disorder (OCD), generalized anxiety order (GAD), post-traumatic stress disorder (PTSD), etc. In such case, the first sensor (e.g., a non-invasive scalp electrophysiological sensor 104a) and second sensor (e.g., a non-invasive physiological sensor 104b) are configured to detect respective correlates of brain signals related to impulsivity and anxiety based disorders.

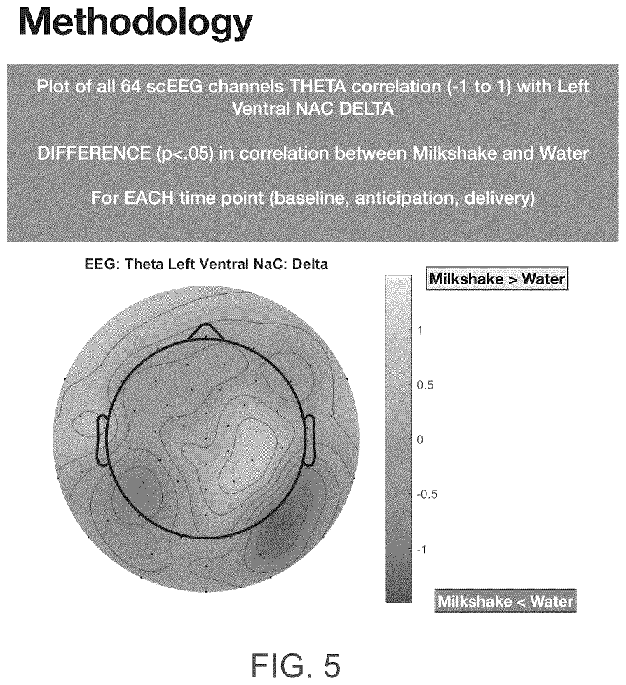

In one embodiment, the wearable, real-time, cognitive behavioral detection system 100 may be specifically configured to treat patients with Loss of Control (LOC) eating disorders. LOC is a component of impulsivity. Empirical invasive experimentation has identified that the low-frequency (1-4 Hz) delta band signal of the NAc is a sensitive measure to impulsivity behaviors causing eating disorders. (see Closing the loop publication and PCT publication WO2018/064225). Referring now to FIGS. 4-9, additional experiments have validated the specificity of the non-invasive target, namely the theta signal (4-8 Hz) above the dlPFC, to identify scalp signals that show connectivity patterns over time with the delta signal of the NAc.

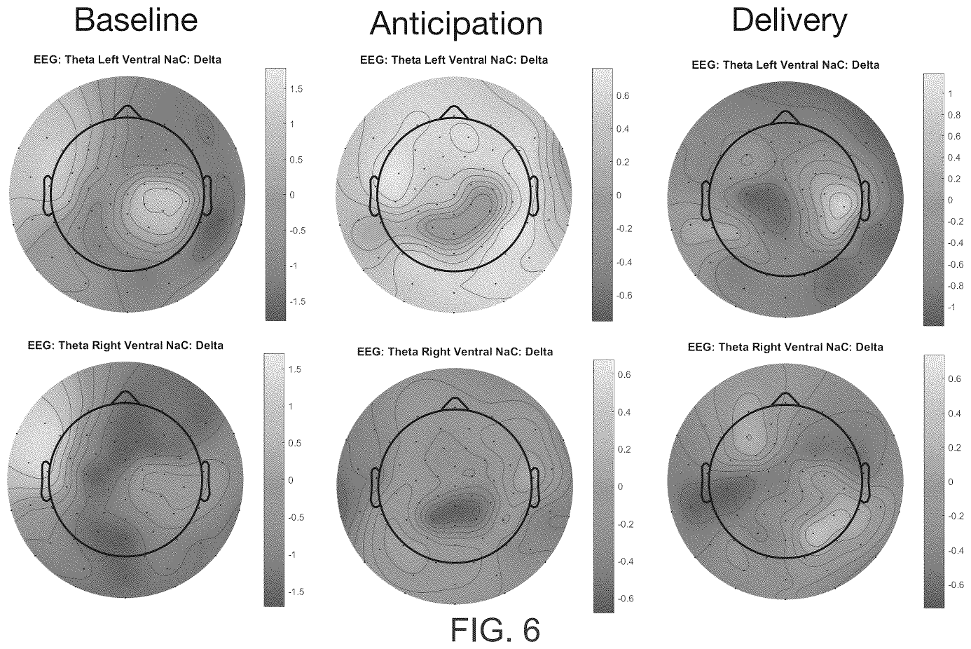

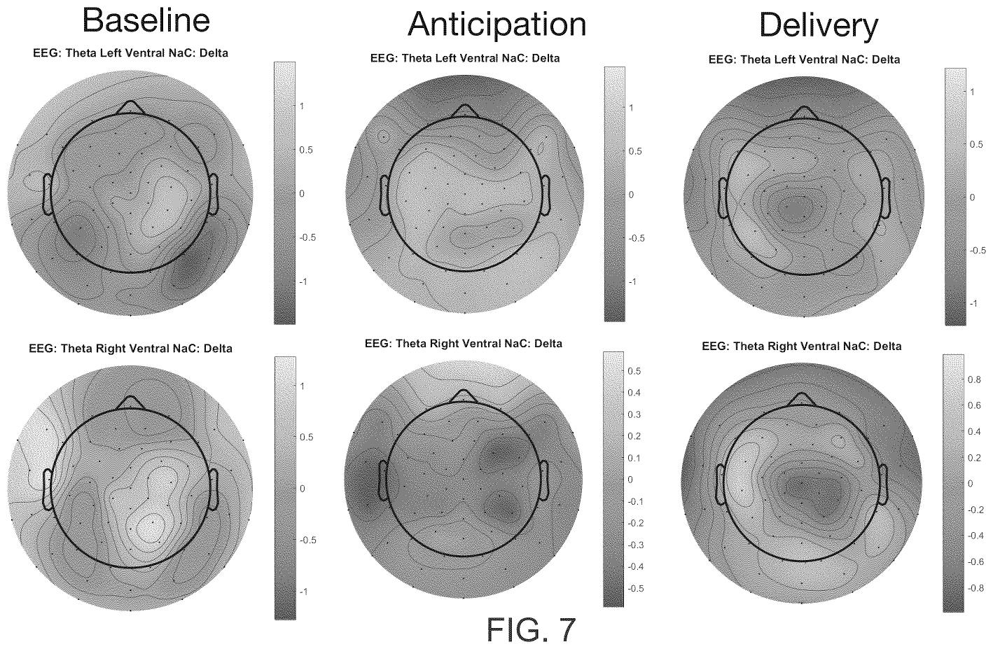

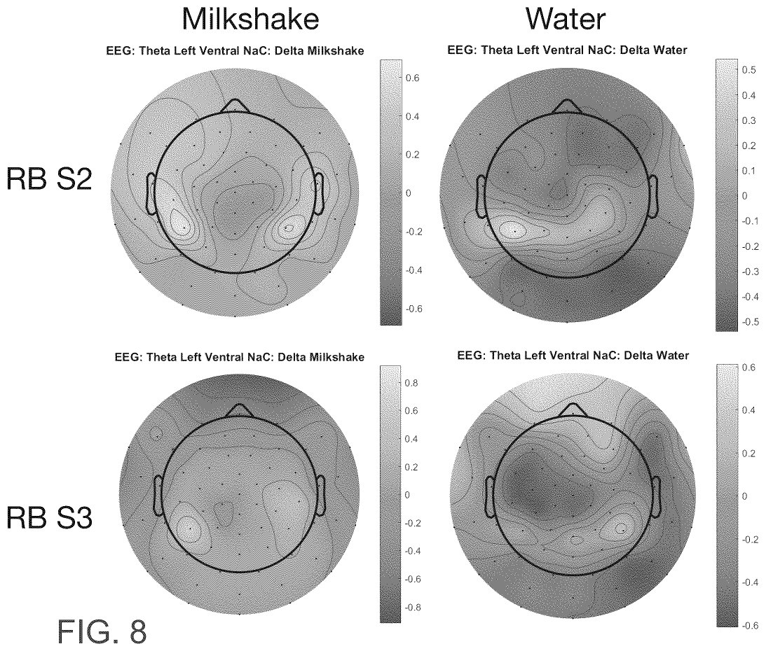

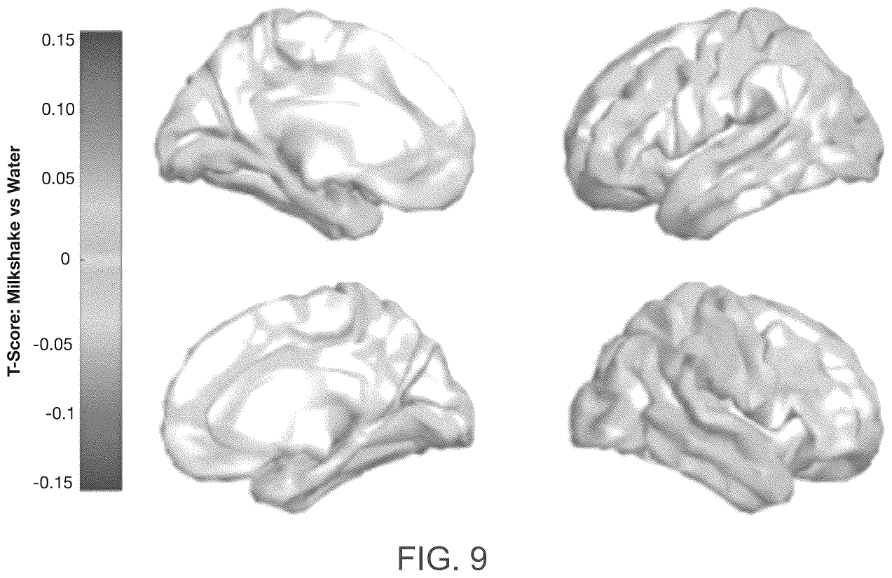

The methodology of the experiments is depicted in FIGS. 4-5. FIG. 4 shows that 80 trials were conducted, and for each trial 64 channels were recorded for a simultaneous scalp electroencephalogram (scEEG) from non-invasive scalp sensors (position on the subject above the dlPFC) and 4 channels were recorded for an intracranial electroencephalogram (iEEG) from intracranial (invasive) sensors directly measuring the delta signal of the NAc. FIG. 5 illustrates experimental results in the form of a plot of all 64 scEEG channels showing that the theta signal (4-8 Hz), as detected by the non-invasive scalp EEG, is a correlate with the invasive delta signal of the left ventral NAc. FIGS. 6-7 also show that the correlation between the non-invasively detected scalp EEG theta signal (4-8 Hz) and the invasive delta signal of the left ventral NAc is significantly elevated during “appetitive” anticipatory state (preceding milkshake), compared to “non-appetitive” anticipatory states (preceding water). These results show this signal is specific to the anticipatory response, and not found in either the baseline (prior to anticipatory cue) nor delivery (receival of anticipated fluid) states. FIG. 8 illustrates the experimental results for a test subject whose preference switched to no longer favor water over milkshake. FIG. 8 shows that the anticipatory elevation is not seen in the dlPFC theta signal specific to “appetitive” state. This further indicates that the non-invasively detected dlPFC theta signal is specific to the LOC eating disorder (i.e., impulsivity), as opposed to the general “appetitive” state. FIG. 9 illustrates a T-Score comparison of the “appetitive” anticipatory state preceding milkshake compared to water. Accordingly, in one embodiment of the cognitive behavioral system 100, the scalp electrophysiological sensor 104a is configured to detect the dlPFC Theta signal correlate of the delta band of NAc signal.

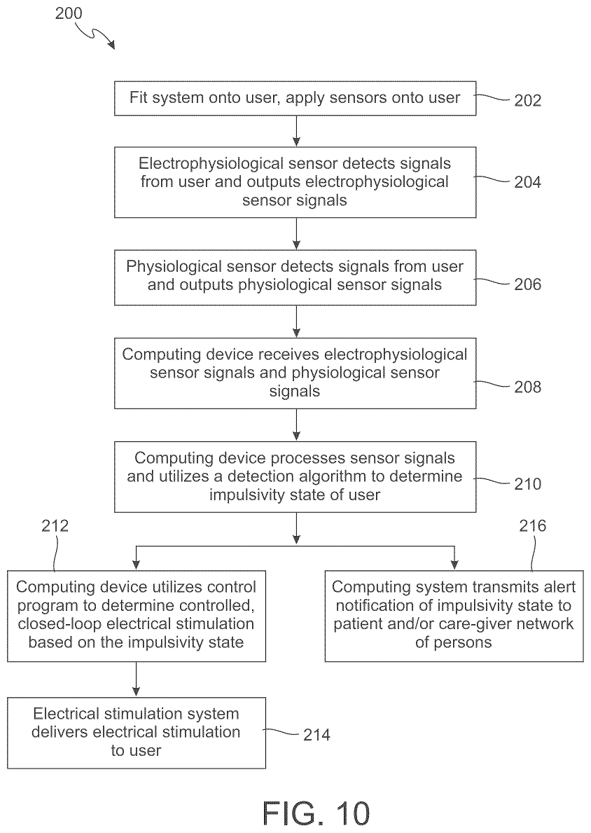

Referring now to FIG. 10, a flow chart illustrates an exemplary method 200 of using the wearable, real-time, cognitive behavioral system 100 for detecting and/or treating impulsivity states of a user 102. At step 202, the cognitive behavioral system 100 is fitted onto the user 102. The scalp electrophysiological sensor 104a is applied to the scalp of the user 102 above the dlPFC. The physiological sensor 104b is applied to the user 102 at one or more locations other than the scalp. At step 204, the scalp electrophysiological sensor 104a detects electrophysiological signals from the user 102 and outputs electrophysiological sensor signals 110a corresponding to the detected electrophysiological signals. At step 206, the physiological sensor 104b detects physiological signals from the user 102 and outputs a physiological sensor signal 110b corresponding to the detected physiological signal. At step 208, the computing device 120 receives the electrophysiological sensor signals 110a and physiological sensor signals 110b. At step 210, the computing device 120 processes the electrophysiological sensor signals 110a and physiological sensor signals 110b, and utilizes a detection algorithm to detect an impulsivity state of the user based on the electrophysiological sensor signals 110a and physiological sensor signals 110b. A impulsivity state is detected when the two input signals—physiologic (e.g., heart rate) and electrophysiologic (e.g., theta power), together exceed an experimentally-defined amplitude threshold. At optional step 212, the computing device 120 utilizes a control program to control the electrical stimulation system 140 to deliver a controlled, closed-loop electrical stimulation 142 to the user 102 based on the impulsivity state. At optional step 214, the electrical stimulation system 142 delivers the controlled, closed-loop electrical stimulation 142 directly to the user 102. In addition, or alternative to determining a delivering electrical stimulation treatment, the method 200 may include a step 216 in which the computing system 120 transmits an alert notification 134 regarding the detected impulsivity state to the patient 102 and/or the network of caregivers 136.

In other embodiments, and as described herein, the method 200 for using the wearable, real-time, cognitive behavioral therapy system 100 and/or device 101 may be configured to detect and/or treat any suitable impulsivity and anxiety based disorders, such as LOC eating disorders, obsessive compulsive disorder (OCD), generalized anxiety order (GAD), post-traumatic stress disorder (PTSD), etc.

While the embodiments are susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

What is claimed is:

1. A system, comprising:
a first sensor configured to be worn on a user, the first sensor being a non-invasive sensor configured to detect an electrophysiology signal at the scalp of the user and to output a first sensor signal corresponding to the detected electrophysiology signal;
a second sensor configured to be worn on the user, the second sensor being a non-invasive sensor configured to detect a physiological signal at a body location other than the scalp and to output a second sensor signal corresponding to the detected physiological signal.
a computing device in operable communication with the first sensor and second sensor, the computing device configured to receive the first sensor signal and second sensor signal, the computing device having a software application which programs the computing device to process the first and second sensor signals and utilize a detection algorithm which utilizes both the first sensor signal and the second sensor signal to detect an impulsivity state of the user. An impulsivity state is detected when the two input signals—physiologic (e.g., heart rate) and electrophysiologic (e.g., theta power), together exceed an experimentally-defined amplitude threshold. Both signals would be transmitted and processed in a real-time fashion. The physiologic signal would be extracted to quantify heart rate, breathing rate and galvanic skin response. The electrophysiologic signal would be extracted to quantify the spectral power from 4-50 Hz, in 1 Hz increments. The detection threshold would be pre-defined experimentally from a set of prior experimentation and validations, and would present as some X percent increase in amplitude in a 2-second instantaneous signal capture above a moving 2-minute baseline average for all incorporated signals. The signals contributing to this detection algorithm would be experimentally defined based on those signals most relevant to the state being detected.

2. The system of claim 1, wherein the electrophysiology signal is a correlate of a nucleus accumbens (NAc) signal.

3. The system of claim 2, wherein the first sensor is a scalp sensor comprising an array of scalp sensors which detects a dorsal-lateral prefrontal cortex (dlPFC) theta (4-8 Hz) signal.

4. The system of claim 1, wherein the physiological signal is a correlate of a nucleus accumbens (NAc) signal.

5. The system of claim 4, wherein the second sensor is a heart rate sensor configured to detect at least one of heart rate and heart rate variability.

6. The system of claim 4, wherein the second sensor comprises one or more electrodes configured to be placed above the user's wrist to detect at least one of heart rate and heart rate variability.

7. The system of claim 1, wherein the first sensor is configured to detect a correlate of the delta band of a nucleus accumbens (NAc) signal.

8. The system of claim 7, wherein the correlate of the delta band of NAc signal is a dorsal-lateral prefrontal cortex (dlPFC) theta (4-8 Hz) signal.

9. The system of claim 1, wherein the detection algorithm is validated for specificity, sensitivity and reliability in detecting the impulsive state by utilizing both the electrophysiological signal at the scalp and the physiological signal at a location other than the scalp.

10. The system of claim 1, wherein the computing device is configured to communicate with the first sensor and second sensor via a wireless communication protocol.

11. The system of claim 10, wherein the wireless communication protocol is one of Bluetooth, WiFi, and wireless USB.

12. The system of claim 1, further comprising:
an electrical stimulation system configured to deliver electrical stimulation directly to the user in response to the impulsivity state; and
wherein the software application is configured to utilize a control program to control the electrical stimulation system to deliver a controlled, closed-loop electrical stimulation to the user based on the impulsivity state.

13. The device of claim 12, wherein the impulsivity state is a loss of control eating behavior, and the electrical stimulation system is configured to deliver electrical stimulation to the nucleus accumbens (NAc) of the user configured to attenuate the loss of control eating behavior.

14. The device of claim 13, wherein the electrical stimulation system is a closed-loop stimulation system.

15. A method of determining an impulsivity state of a user, comprising:
obtaining a first sensor signal corresponding to a detected electrophysiology signal from a first sensor worn on the scalp of the user;
obtaining a second sensor signal corresponding to a detected physiological signal from a second sensor positioned at a body location of the user other than the scalp;
a computing device receiving the first sensor signal and the second sensor signal;
the computing device processing the first sensor signal and the second sensor signal and detecting an impulsivity state of the user utilizing a detection algorithm which utilizes both the first sensor signal and the second sensor signal.

16. The method of claim 15, wherein the electrophysiology signal is a correlate of a nucleus accumbens (NAc) signal.

17. The method of claim 16, wherein the first sensor is a scalp sensor comprising an array of scalp sensors which detects a dorsal-lateral prefrontal cortex (dlPFC) theta (4-8 Hz) signal.

18. The method of claim 15, wherein the physiological signal is a correlate of a nucleus accumbens (NAc) signal.

19. The method of claim 4, wherein the second sensor is a heart rate sensor which detects at least one of heart rate and heart rate variability.

20. The method of claim 4, wherein the second sensor comprises one or more electrodes placed above the user's wrist to detect at least one of heart rate and heart rate variability.

21. The method of claim 15, wherein the first sensor is configured to detect a correlate of the delta band of a nucleus accumbens (NAc) signal.

22. The method of claim 21, wherein the correlate of the delta band of NAc signal is a dorsal-lateral prefrontal cortex (dlPFC) theta (4-8 Hz) signal.

23. The method of claim 15, wherein the detection algorithm is validated for specificity, sensitivity and reliability in detecting the impulsive state by utilizing both the electrophysiological signal at the scalp and the physiological signal at other than the scalp.

24. The method of claim 15, wherein the computing device is configured to communicate with the first sensor and second sensor via a wireless communication protocol.

25. The method of claim 24, wherein the wireless communication protocol is one of Bluetooth, WiFi, and wireless USB.

26. The method of claim 15, further comprising:
a software application utilizing a control program to control an electrical stimulation system to deliver a controlled, closed-loop electrical stimulation to the user based on the impulsivity state; and
the electrical stimulation system delivering the controlled, closed-loop electrical stimulation directly to the user in response to the impulsivity state.

27. The method of claim 26, wherein the impulsivity state is a loss of control eating behavior, and the electrical stimulation system delivers electrical stimulation to the nucleus accumbens (NAc) of the user configured to attenuate the loss of control eating behavior.

28. The method of claim 27, wherein the electrical stimulation device is a closed-loop system.

29. The method of claim 15, wherein the impulsivity state of the user is a state in which the user has a craving.

30. A computer executable method stored on a storage device, comprising:
receiving as input a first sensor signal or information indicative of the first second signal, the first sensor signal corresponding to a detected electrophysiology signal from a first sensor worn on the scalp of the user;
receiving as input a second sensor signal or information indicative of the second signal, the second sensor signal corresponding to a detected physiological signal from a second sensor positioned at a body location of the user other than the scalp; and
determining an impulsivity state of the user utilizing the first sensor signal and the second sensor signal, or the information indicative of the first and second sensor signals.

31. The method of claim 30, wherein determining the impulsivity state of the user comprises utilizing a threshold.
